(19) United States
(12) Patent Application Publication
(10) Pub. No.: US 2024/0252767 A1
(43) Pub. Date: Aug. 1, 2024

(54) DEVICES AND METHODS FOR BENDING A MEDICAL OR SURGICAL INSTRUMENT

(71) Applicant: THE REGENTS OF THE UNIVERSITY OF CALIFORNIA, Oakland, CA (US)

(72) Inventors: Scott Mitchell Fishman, Sacramento, CA (US); Nathan Galen Rosecrans, Sacramento, CA (US)

(21) Appl. No.: 18/563,454

(22) PCT Filed: May 25, 2022
(86) PCT No.: PCT/US22/30801

§ 371 Date: Nov. 22, 2023

Related U.S. Application Data
(60) Provisional application No. 63/193,465, filed on May 26, 2021.

(51) Int. Cl. A61M 5/32

(52) U.S. Cl. CPC A61M 5/329; A61M 2207/10; A61M 2209/04

(57) ABSTRACT

Devices and methods for bending a medical or surgical instrument are disclosed. A device may include a handle and a bending die. A first end of the bending die may be coupled to the handle. A recess may be formed partially or entirely in a second end of the bending die. The recess may be configured to permit a portion or an entirety of the medical or surgical instrument to be inserted laterally into the bending die. A method may include laterally inserting the medical or surgical instrument into the bending die and rotating the bending die with respect to the medical or surgical instrument to press the medical or surgical instrument against the bending die to form one or more bends in the medical or surgical instrument.

### CROSS-REFERENCE TO RELATED APPLICATION

This application claims priority to U.S. Provisional Application No. 63/193,465, filed May 26, 2021, the disclosure of which is herein incorporated by reference in its entirety for all purposes.

### FIELD OF DISCLOSURE

The present application generally relates to imparting one or more desired bends to a pliable article, and, more particularly, devices and methods for bending medical or surgical instruments.

### BACKGROUND

A user of a medical or surgical instrument may in certain instances find it necessary to bend the instrument, for example, to customize it for use in a particular procedure or with a particular patient. Hypodermic needles are an example of a medical or surgical instrument for which is may be advantageous to create one or more strategically placed bends. The bend(s) may, for example, facilitate maneuvering the needle to avoid intervening nerves, blood vessels, organs, and/or other tissue that would otherwise come into contact with the tip of the needle if it followed a strictly linear path into the body toward a desired treatment site. The location of the bend, its angle, the number of bends, and/or other characteristics of the bend(s) may depend on the medical or surgical procedure at hand, the anatomy of the patient, the gauge and/or type of needle, and/or other variables. As a consequence, a needle with a preformed bend may not be useful depending on the intended use of the needle. Instead, a user may wish to bend the needle at the point of care or use, where the variable(s) affecting what is an optimal bend can be assessed and properly accounted for.

Bending a needle or other medical or surgical instrument in a point-of-care setting or other point-of-use setting can involve certain challenges. For example, if a user must manually bend the instrument in his or her hands, it may be difficult to form a bend having a precise angle or curvature and/or to create multiple bends having uniform angles and/or curvatures. Additionally, a purely manual technique tends to be slow, inefficient, and/or may require increased handling that, in certain instances, may increase the risk of inadvertently compromising a sterile or aseptic condition of the instrument. Further, in the case of bending a hollow needle, it is possible that a user may inadvertently crush the lumen of the needle, blunt the tip of the needle, and/or cause other structural damage as a result of, for example, applying excessive force to the needle.

The present disclosure sets forth devices and methods embodying advantageous alternatives to existing devices and methods for bending medical and surgical instruments that address one or more of the challenges or needs mentioned herein, as well as provide other benefits and advantages.

### SUMMARY

One aspect of the present disclosure provides a device for bending a medical or surgical instrument. The device may include a handle and a bending die. A first end of the bending die may be coupled to the handle. A first recess may be formed partially or entirely in a second end of the bending die. The first recess may be configured to permit a portion or an entirety of the medical or surgical instrument to be inserted laterally into the bending die. Moreover, the first recess may configured to permit the medical or surgical instrument to be inserted into the bending die in a direction that is perpendicular or otherwise transverse to a longitudinal axis of the medical or surgical instrument. In some embodiments, the medical or surgical instrument may correspond to a needle such as hypodermic needle.

A second recess may be formed partially or entirely in the second end of the bending die. The first recess may cooperate with the second recess such that the medical or surgical instrument can be inserted into the second recess by way of the first recess. The first recess may intersect with the second recess at an angle that is less than or equal to 90 degrees. The second end of the bending die may include a bending surface disposed at least partially at a bottom of the second recess. The bending surface may be configured to impart at least one bend to the medical or surgical instrument during use of the device. A portion or the entirety of the bending surface may possess a convex curvature.

The second end of the bending die may also include one or more rolling surfaces configured to roll against an external object to assist in causing the medical or surgical instrument to conform to a shape of the bending surface during use of the device. A portion or the entirety of the one or more rolling surfaces may have a convex curvature. At least a portion of at least one of the rolling surfaces may possess a curvature that is identical or substantially similar to a curvature of at least a portion of the bending surface.

The bending die may also include a first wall, a second wall, a third wall, and/or a fourth wall extending between the first and second walls. At least a portion of or the entirety of the first recess may disposed between the first wall and the third wall. At least a portion of or the entirety of the second recess may be disposed between the second wall and the third wall. An opening may be formed partially or entirely in the fourth wall and/or may communicate with (e.g., open into) the first recess and/or the second recess.

Additional optional aspects of the device include the following. The handle and the bending die may be integrally formed to define a single unitary structure, or, alternatively, may be separate interconnected structures. The bending die may include a plurality of markings for assisting a user in bending the medical or surgical instrument. The bending die may be provided to a user in a sterile or aseptic condition. The handle may be configured to held in at least one hand of the user.

Another aspect of the present disclosure provides a method of bending a medical or surgical instrument. The method may include: providing a device having a handle and a bending die; laterally inserting the medical or surgical instrument into the bending die; and rotating the bending die with respect to the medical or surgical instrument to press the medical or surgical instrument against the bending die to form one or more bends in the medical or surgical instrument. Laterally inserting the medical or surgical instrument into the bending die may include translating the medical or surgical instrument in a direction transverse to a longitudinal axis of the medical or surgical instrument. A user may use the handle to manually rotate the bending die with respect to the medical or surgical instrument, or, alternatively, a machine may use the handle to automatically or semi-automatically rotate the bending die with respect to the medical or surgical instrument.

Rotating the bending die with respect to the medical or surgical instrument may include rolling the bending die against an external object.

The method may include translating the medical or surgical instrument with respect to the bending die to position the medical or surgical instrument in a first predetermined bending position with respect to the bending die, for example, after or simultaneous with laterally inserting the medical or surgical instrument into the bending die and/or prior to or simultaneous with forming the first bend.

After forming the first bend in the medical or surgical instrument, the method may include: (a) translating the medical or surgical instrument with respect to the bending die to position the medical or surgical instrument in a second predetermined bending position with respect to the bending die; and (b) subsequent to or simultaneous with (a), further rotating the bending die with respect to medical or surgical instrument to press the medical or surgical instrument against the bending die to form a second bend in the medical or surgical instrument. At least a portion of the medical or surgical instrument may remain disposed within the bending die during an entire time between forming the first bend and forming the second bend.

In embodiments where the medical or surgical instrument is a needle, the method may include, subsequent to or simultaneous with laterally inserting the hypodermic needle into the bending die and prior to or simultaneous with forming the first bend, rotating the hypodermic needle about a longitudinal axis of the hypodermic needle to position a bevel of the hypodermic needle in a predetermined bevel position with respect to the to-be-formed first bend.

The method may be performed partially or entirely in a point-of-care setting such as, for example, an operating room, a hospital room, a doctor's office, a field hospital, and the like. Further, the medical or surgical instrument may be used in a medical or surgical procedure performed on a patient substantially immediately (e.g., within seconds or minutes) after the medical or surgical instrument is bent using the bending die.

### BRIEF DESCRIPTION OF THE DRAWINGS

It is believed that the disclosure will be more fully understood from the following description taken in conjunction with the accompanying drawings. Some of the drawings may have been simplified by the omission of selected elements for the purpose of more clearly showing other elements. Such omissions of elements in some drawings are not necessarily indicative of the presence or absence of particular elements in any of the exemplary embodiments, except as may be explicitly delineated in the corresponding written description. Also, none of the drawings are necessarily to scale.

FIG. 1 is a perspective view of a device for bending a medical or surgical instrument according to an embodiment of the present disclosure.

FIG. 2 is a perspective view of an end of the device in FIG. 1.

FIG. 3 is another perspective of the end of the device in FIG. 1.

FIG. 4 is a top plan view of the device in FIG. 1.

FIG. 5 is a bottom plan view of the device in FIG. 1.

FIG. 6 is a side view of the device in FIG. 1.

FIG. 7 is a perspective view of a portion of a medical or surgical instrument.

FIG. 8 is a side view of the portion of the medical or surgical instrument in FIG. 7.

FIGS. 9-14 illustrate steps of a method, in chronological order, of a method for bending a medical or surgical instrument according to an embodiment of the present disclosure.

FIGS. 15 and 16 illustrate steps of a method, in chronological order, of a method for bending a medical or surgical instrument according to another embodiment of the present disclosure.

### DETAILED DESCRIPTION

Generally the present disclosure relates to devices and methods for bending medical and surgical instruments. The devices and methods described herein have various advantages including allowing one to bend a medical or surgical instrument in a point-of-care setting or other point-of-use setting. One may use the presently disclosed devices and methods to adapt or customize a medical or surgical instrument for a particular application such as a particular medical or surgical procedure or technique and/or to accommodate the anatomy and/or preferences of a particular patient and/or user. As an example, the presently disclosed devices and methods may be used to form one or more strategically placed bends in a needle such as a hypodermic needle. As a more specific example, the presently disclosed devices and methods may allow a surgeon, surgical assistant, and/or other user to create one or more identical or different bends in one or more needles during or substantially immediately prior to use of the needle(s) in surgery. As an even more specific example, the presently disclosed devices and methods may allow a user to laterally insert a needle into a bending die, thereby eliminating or substantially reducing the risk damage to and/or contamination of a tip of the needle, and, furthermore, improving visibility of the tip of the needle so that the user is able to visually confirm that the needle is bent in a desired direction toward or away from a bevel or other structure included at the tip of the needle. The precision, reproducibility, ease-of-use, and other advantageous characteristics of the presently disclosed devices and methods may generally have the effect of improving the quality and efficiency of medical care. Additional benefits, advantages, and uses of the disclosed devices and methods are possible and are not limited to those mentioned herein.

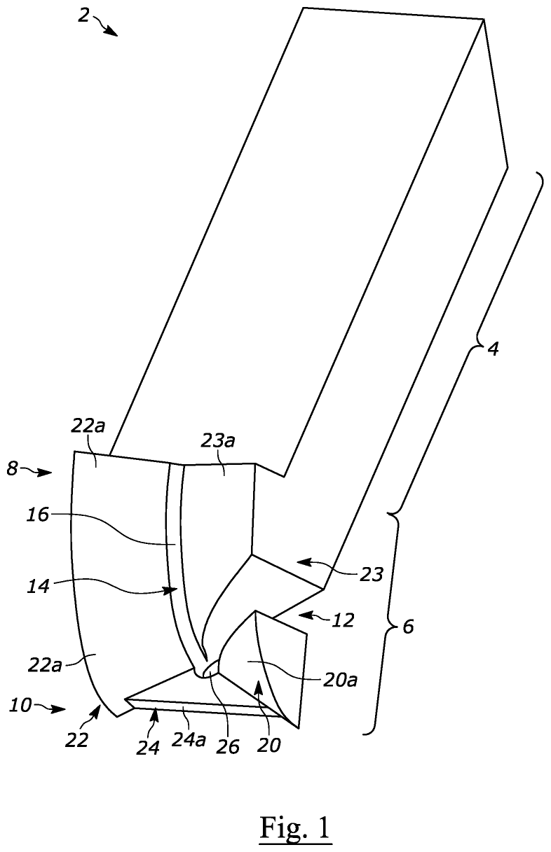

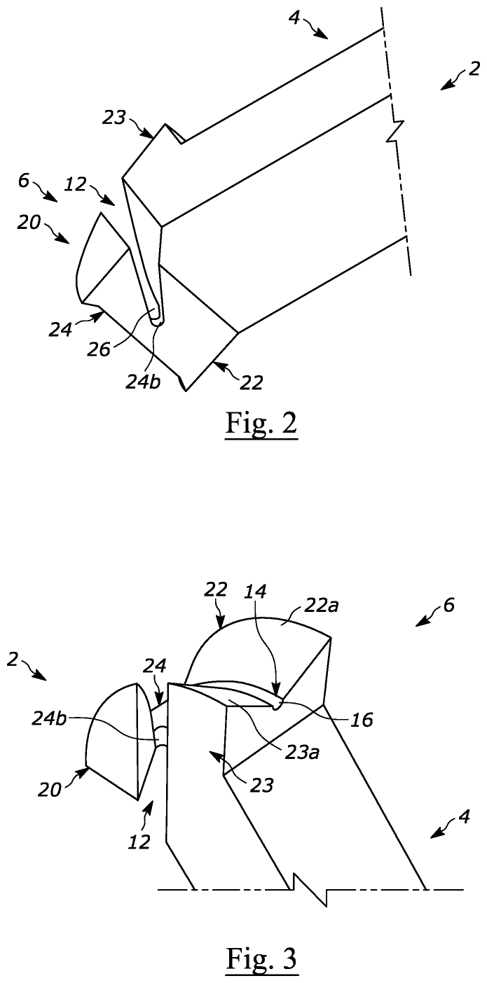

FIGS. 1-6 illustrate a tool or device 2 for bending a medical or surgical instrument according to an embodiment of the present disclosure. The device 2 may be configured to bend a medical or surgical instrument having a generally elongated shape including, but not limited to, a needle (e.g., a hypodermic needle, a trocar needle, a hollow needle, a solid needle), a cannula, a catheter, a guidewire, and/or any combination thereof, including, for example, a needle disposed at least partially within a lumen of a cannula. The medical or surgical instrument may be constructed partially or entirely of metal and/or any other generally pliable material. The device 2 may be provided to a user in a sterile or aseptic condition. For example, the device 2 may be provided in sterile or aseptic packaging that is removed by the user prior to use. The sterile or aseptic condition of the device 2 may prevent or inhibit the device 2 from passing contaminants to the medical or surgical instrument during use. The user of the device 2 can be any person or persons including, but not limited to, any healthcare practitioner including, for example, a surgeon, a physician, a nurse, and/or a surgical assistant, as well as other healthcare professionals. Additionally or alternatively, the user may be an automated or semi-automated machine including, for example, a surgical robot.

Generally the device 2 may include a handle 4 and a bending die 6. The bending die 6 may have a first end 8 coupled to the handle 4 and a second end 10 defining a terminal or free end of the device 2. As an example, the handle 4 may be rigidly or fixedly coupled to the first end 8 of the bending die 6 such that the handle 4 does not move independently of or with respect to the first end 8 of the bending die 6. The terms “coupled,” “connected,” and the like as used herein mean the joining of two members directly or indirectly to one another. Such joining may be stationary (e.g., permanent, fixed, and/or rigid) or moveable (e.g., removable, releasable, flexible, and/or including a mechanical linkage with moving part(s)). Furthermore, such joining may be achieved with the two members, or the two members and any additional intermediate members, being integrally formed with one another to define as a single unitary structure, or with the two members, or the two members and any additional intermediate members, being separate, distinct structures attached to one another.

The device 2 may be constructed of rigid material including, for example, metal (e.g., stainless steel and/or surgical steel), plastic, ceramic, or any other suitable material, or any combination thereof. In some embodiments, a material used to construct the device 2, or at least the bending die 6, may be more rigid than a material used to construct the medical or surgical instrument or at least the portion of the medical or surgical instrument to be bent with the device 2.

The handle 4 generally may be configured to provide a user with mechanical leverage when bending the medical or surgical instrument with the bending die 6. As an example, the handle 4 may be configured as a lever for amplifying a force applied by the user to the handle 4. A length of the handle 4 may be chosen depending on, for example, an amount of force capable of being applied by the user and/or an amount of force needed for bending a particular medical or surgical instrument. The length of the handle 4 may be proportional (e.g., directly proportional) to the amount of force that the bending die 6 applies to the medical or surgical instrument during use of the device 2. As an example, an increase in the length of the handle 4 may cause a corresponding increase in the amount of force applied by the bending die 6 to the medical or surgical instrument. In some embodiments, the handle 4 may be ergonomically sized, shaped, textured, and/or dimensioned to be held and/or gripped by one or more or hands of a person including, for example, one or more fingers of the person. Additionally or alternatively, the handle 4 may configured to be coupled to an external device or machine. In some embodiments, the handle 4 may be omitted.

The bending die 6 generally may be configured to press one or more portions of the medical or surgical instrument against one or more external objects to form one or more bends in the medical or surgical instrument. As an example, the bending die 6 may be configured to press the medical or surgical instrument against an external object such that the medical or surgical instrument conforms to a shape (e.g., a curvature and/or angle) of the bending die 6 and/or the external object. The external object may include, for example, a generally planar surface such as a surgical tray, a surgical table, a tabletop, a countertop, and the like. Additionally or alternatively, the external object may be a user's hand, such as the user's hand which is not being used hold the handle 4. Pressing the medical or surgical instrument between the bending die 6 and the external object may involve moving the bending die 6 toward the external object while the external object remains stationary, moving the external object toward the bending die 6 while the bending die 6 remains stationary, or moving both the bending die 6 and the external object towards each other. As an example, the bending die 6 may be configured to rotate with respect to the external object and/or the medical or surgical instrument while the medical or surgical instrument is pressed in between the bending die 6 and the external object, thereby causing the bending die 6 to impart one or more bends to the medical or surgical instrument. As a more specific example, the bending die 6 may be configured to roll against the external object with the medical or surgical instrument disposed there between in order to form one or more bends.

A width of the bending die 6 may be greater than a corresponding width of the handle 6 such that one or more sides of the bending die 6 overhang corresponding side(s) of the handle 6, as seen in FIG. 6. This configuration may increase the mechanical leverage that the handle 4 is able to provide during bending.

The bending die 6 may have one or more recesses sized to receive at least a portion of the medical or surgical instrument. As used herein, the term “recess” refers to any portion of a wall or other structure that is set back with respect to an adjacent portion of the wall or structure and encompasses without limitation a groove, a slot, a depression, a channel, an opening, a hole, a through hole, a blind hole, and the like. Referring to FIG. 1, a first recess 12 and a second recess 14 may be formed in the second end 10 of the bending die 6. The first recess 12 may be configured to permit at least a portion of the medical or surgical instrument to be inserted laterally into the bending die 6. As an example, laterally inserting the medical or surgical instrument into the first recess 12 may involve moving the medical or surgical instrument in a direction that is perpendicular to or otherwise transverse to a longitudinal axis of the medical or surgical instrument such that a lateral side of the medical or surgical instrument is initially received within the first recess 12. As a more specific example, laterally inserting the medical or surgical instrument into the first recess 12 may involve inserting the medical or surgical instrument in a non-axial direction into the first recess 12. In an embodiment where the medical or surgical instrument is a needle, laterally inserting the needle into the first recess 12 may involve initially inserting a non-tip portion of the needle into the first recess 12. As a consequence, the tip of the needle may remain visible while the needle is inserted into the first recess 12, for example, without the bending die 6 obstructing the tip of the needle from view of the user. This may allow the user to arrange a bevel or other structure at the tip of the needle in a desired position (e.g., a desired axial and/or rotational position) with respect to the to-be-formed bend(s), as described below in more detail.

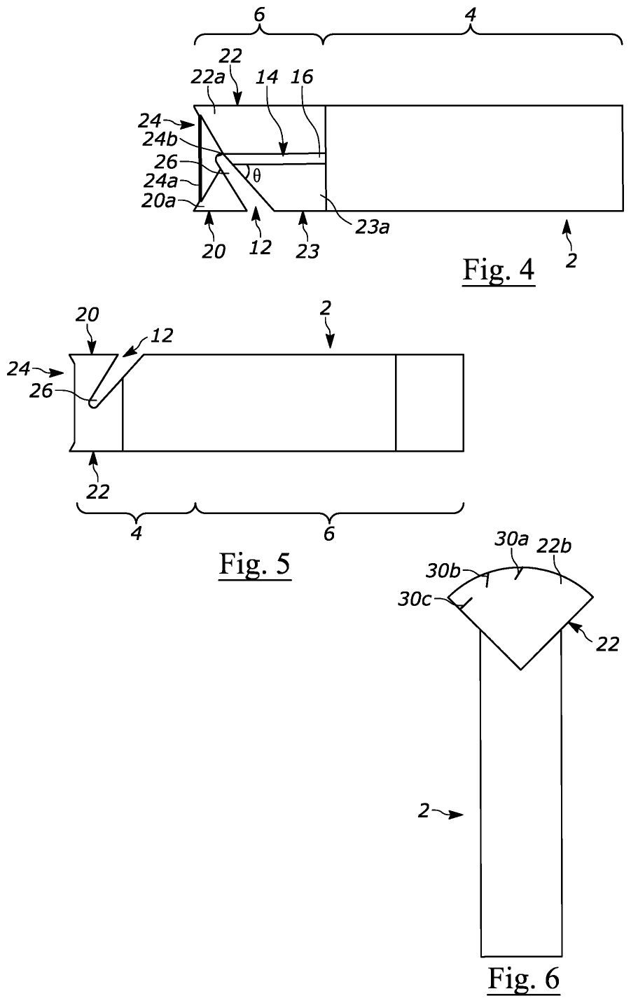

The first recess 12 may cooperate with (e.g., adjoin, intersect with, and/or communicate with) the second recess 14 such that the medical or surgical instrument can be inserted into the second recess 14 by way of the first recess 12. As an example, the first recess 12 may open into the second recess 14 such that inserting the medical or surgical instrument into the bending die 6 involves inserting the medical or surgical instrument through the first recess 12 and subsequently into the second recess 14. A surface defining the first recess 12 may intersect with a surface (e.g., the bending surface 16) defining the second recess 14 at an angle θ, as shown in FIG. 4. As an example, the angle θ may be less than 180 degrees, or less than or equal to approximately (e.g., ±10%) 135 degrees, or less than or equal to approximately (e.g., ±10%) 90 degrees, or less than or equal to approximately (e.g., ±10%) 75 degrees, or less than or equal to approximately (e.g., ±10%) 60 degrees, or less than or equal to approximately (e.g., ±10%) 45 degrees, or less than or equal to approximately (e.g., ±10%) 30 degrees, or less than or equal to approximately (e.g., ±10%) 15 degrees, or any other suitable range or specific numerical amount.

At least in embodiments where the medical or surgical instrument is a needle or similar, a width of the first recess 12 and/or a width of the second recess 14 may be less than or equal to approximately (e.g., ±10%) 20 mm, or less than or equal to approximately (e.g., ±10%) 15 mm, or less than or equal to approximately (e.g., ±10%) 10 mm, or less than or equal to approximately (e.g., ±10%) 7.5 mm, or less than or equal to approximately (e.g., ±10%) 5 mm, or any other suitable range or specific numerical amount. As seen in FIG. 4, a width of the first recess 12 may gradually decrease or taper in a direction moving toward the second recess 14 to assist with guiding the medical or surgical instrument toward the second recess 14 during insertion of the medical or surgical instrument into the bending die 6.

Referring to FIGS. 1, 3, and 4, the second end 10 of the bending die 6 may include a bending surface 16 disposed at a bottom of the second recess 14. The bending surface 16 or at least a portion thereof may generally face in a direction away from the handle 4. The bending surface 16 may be configured to impart one or more bends to the medical or surgical instrument when the medical or surgical instrument is pressed against the bending surface 16. The bending surface 16 may have any suitable shape to achieve the desired bend(s). As an example, the bending surface 16 may a have a non-linear shape such as a curved shape or a curvilinear shape. As a more specific example, the bending surface 16 may follow an arc (or arcs) that is centered about an axis that is parallel to or otherwise non-perpendicular to, or coaxial with, an axis around which the bending die 6 is rotated during bending. A curvature of the bending surface 16 may be constant along an entire length of the bending surface 16 or vary along the length of the bending surface 16. In some embodiments, a radius of curvature of a portion of or the entirety of the bending surface 16 may be less than or equal to approximately (e.g., ±10%) 100 mm, or less than or equal to approximately (e.g., ±10%) 75 mm, or less than or equal to approximately (e.g., ±10%) 50 mm, or less than or equal to approximately (e.g., ±10%) 25 mm, or any other suitable range or specific numerical amount. In addition to or as an alternative to being curved, the bending surface 16 or a portion thereof may have a linear or planar shape so as to impart a corner-like bend to the medical or surgical instrument. As an example, the bending surface 16 may be configured to impart a bend to the medical or surgical instrument having an angle equal to or less than approximately (e.g., ±10%) 90 degrees, or an angle equal to or less than approximately (e.g., ±10%) 75 degrees, or an angle equal to or less than approximately (e.g., ±10%) 60 degrees, or an angle equal to or less than approximately (e.g., ±10%) 45 degrees, or an angle equal to or less than approximately (e.g., ±10%) 30 degrees, or an angle equal to or less than approximately (e.g., ±10%) 15 degrees, or any other suitable range or specific numerical amount.

In the embodiment illustrated in FIGS. 1-6, the bending surface 16 has a generally convex shape. In other embodiments, the bending surface 16 may have a concave shape. In still further embodiments, a portion of the bending surface 16 may have a convex shape and a different portion of the bending surface 16 may have concave shape.

The bending die 6 may include one or more walls. As an example, the bending die 6 may include a first wall 20, a second wall 22, a third wall 23, and/or a fourth wall 24 extending between the first wall 20 and the second wall 22. The first wall 20, the second wall 22, the third wall 23, and the fourth wall 24, or any combination thereof, may be separate interconnected structures, or alternatively, integrally formed parts of a single unitary structure as shown in FIGS. 1-6. A portion or the entirety of the first recess 12 may be disposed between the first wall 20 and the third wall 23. As an example, a portion or the entirety of the first recess 12 may be defined by an inwardly facing surface of the first wall 20 and an inwardly facing surface of the third wall 23. A portion or the entirety of the second recess 14 may be disposed between the second wall 22 and the third wall 23. As an example, a portion or the entirety of the second recess 14 may be defined by an inwardly facing surface of the second wall 22 and an inwardly facing surface of the third wall 23.

The first wall 20, the second wall 22, and the third wall 23 may include, respectively, a rolling surface 20a, a rolling surface 22a, and a rolling surface 23a. Any one or combination of the rolling surfaces 20a, 22a, and 23a may be configured to roll against an external object to assist in causing the medical or surgical instrument to conform to a shape of the bending surface 16 during use of the device 2. As an example, any one or combination of the rolling surfaces 20a, 22a, and 23a may have a curvature (including, e.g., a convex and/or concave curvature) allowing the rolling surface to roll against the external object. As a more specific example, a curvature of any one or combination rolling surfaces 20a, 22a, and 23a may correspond to a curvature of a portion of or the entirety of the bending surface 16. As an even more specific example, a radius of curvature of any one or combination rolling surfaces 20a, 22a, and 23a may be identical to a radius of curvature of a portion of or the entirety of the bending surface 16. The rolling surface 20a and/or the rolling surface 23a may be arranged generally at an angle (e.g., an acute angle) relative to the rolling surface 22a. As a consequence, when viewed from the side, the rolling surface 20a and the rolling surface 22a and/or the rolling surface 23a and the rolling surface 22a may appear to generally form a V-shape.

The fourth wall 24 may have a first end coupled to the rolling surface 20a of the first wall 20 and a second end (opposite to the first end) coupled to the rolling surface 22a of the second wall 22. As an example, the fourth wall 24 may be generally perpendicular to or otherwise transverse to the rolling surface 20a of the first wall 20 and/or the rolling surface 22a of the second wall 22. As seen in FIG. 1, an upper surface 24a of the fourth wall 24 may be positioned below an uppermost portion of each of the rolling surface 20a and the rolling surface 22a so that the fourth wall 24 does not interfere with the rolling function of the rolling surfaces 20a and 22a.

An opening 26 may be formed at least partially in the fourth wall 24. As an example, the opening 26 may be a through hole extending across the fourth wall 24 from one side of the fourth wall 24 to an opposite side of the fourth wall 24. The opening 26 may cooperate with (e.g., adjoin, intersect with, and/or communicate with) the first recess 12 and/or the second recess 14. As an example, the opening 26 may open into the first recess 12 and/or the second recess 14 such that the medical or surgical instrument can pass through the opening 26 coming from or going to the first recess 12 and/or the second recess 14. As a more specific example, the opening 26 may be arranged such that the medical or surgical instrument can be inserted into the opening 26 by way of the first recess 12 and/or such that the medical or surgical instrument can be positioned concurrently in the opening 26 and one or both of the first recess 12 and the second recess 14.

The fourth wall 24 may further include an abutment surface 24b configured to abut against the medical or surgical instrument during bending, for example, to prevent or inhibit the medical or surgical instrument from escaping from the second recess 14 when a bending force is applied to the medical or surgical instrument. As an example, the abutment surface 24b may be configured to abut an upwardly facing surface of the medical or surgical instrument during bending in order to keep or assist with keeping a downwardly facing surface of the medical or surgical instrument pressed against the bending surface 16. The abutment surface 24b may face in a generally downwards direction, whereas the bending surface 16 may face in a generally upwards direction. The terms “downwards” and “upwards” and the like herein are merely intended to describe how the mentioned directions compare to one another; these terms are not necessarily related to a pulling direction of Earth's gravity. In some embodiments, the abutment surface 24b may define at least a portion of the periphery of the opening 26.

With reference to FIG. 6, one or more markings 30a-c may be disposed on a side surface 22b of the second wall 22 of the bending die 6. Each of the markings 30a-c may provide the user with a respective visual reference indicative of a curvature and/or angle formed in the medical or surgical instrument during bending. As an example, the markings 30a-c may be a series of darkened line segments as shown in FIG. 6. The markings 30a-c may be created in any suitable manner including being printed, painted, drawn, and/or adhered to the side surface 22b of the second wall 22 of the bending die 6.

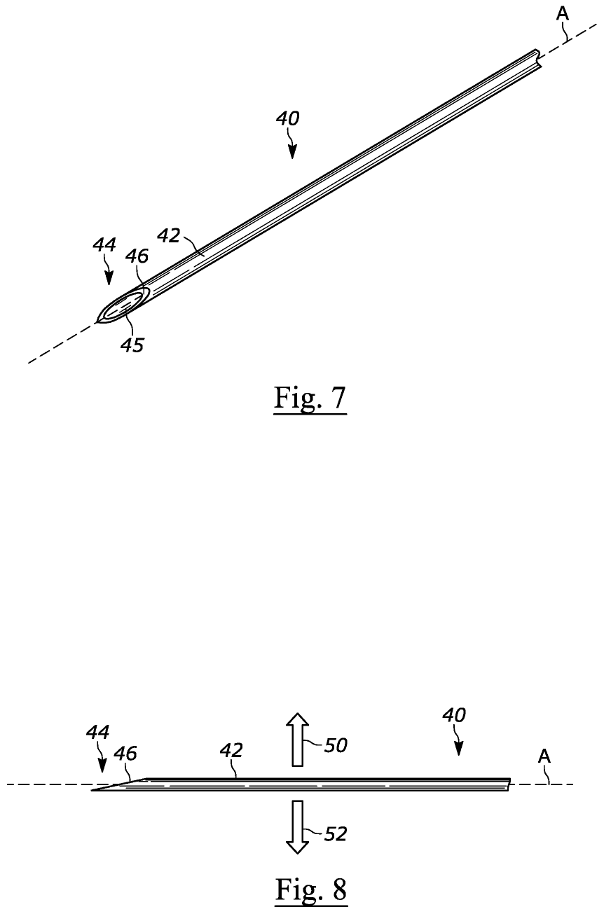

FIGS. 7 and 8 illustrate an end portion (e.g., a distal end portion) of a needle 40 which, according to some embodiments of the present disclosure, may correspond to the medical or surgical instrument (or a portion thereof) that is bent by the device 2. The needle 40 may have longitudinal axis A. As an example, a wall 42 (e.g., an annular or tubular wall) of the needle 40 may be centered around and/or extend along the longitudinal axis A. Prior to or subsequent to bending the needle 40 with the device 2, the longitudinal axis A or at least of portion thereof may be linear. The needle 40 may be hollow such that it has a lumen, as shown in FIG. 7, or, alternatively, the hypodermic needle 40 may be solid such that is has no lumen. The needle 40 may be hypodermic needle or any other type of needle.

In some embodiments, a tip of the needle 40 may include a tapered region 44. As an example, a width of the tapered region 44 may gradually decrease to a point, as shown in FIG. 8. The point may be sharp enough to pierce through, for example, a patient's skin and/or other tissue. An opening 45 or multiple openings may be formed within or adjacent to the tapered region 44 to allow fluid and/or other materials to flow into and/or out of the needle 40. Furthermore, in some embodiments, the tapered region 44 may include a bevel 46. As an example, the bevel 46 may be may be defined by a plane or cut that intersects a first lateral side of the needle 40 at a first axial location along the longitudinal axis A and intersects a second lateral side of the needle 40 at a second axial location along the longitudinal axis A, as seen in FIG. 8. Furthermore, in some embodiments, the tapered region 44 may include multiple bevels formed at different angles relative to one another.

Moving the needle 40 laterally as described herein may involve moving the needle 40 in any direction that is perpendicular to, non-parallel to, or otherwise transverse to the longitudinal axis A of the needle 40. As an example, FIG. 8 includes an arrow 50 identifying movement of the needle 40 in a first lateral direction and an arrow 52 identifying movement of the needle 40 in a second lateral direction.

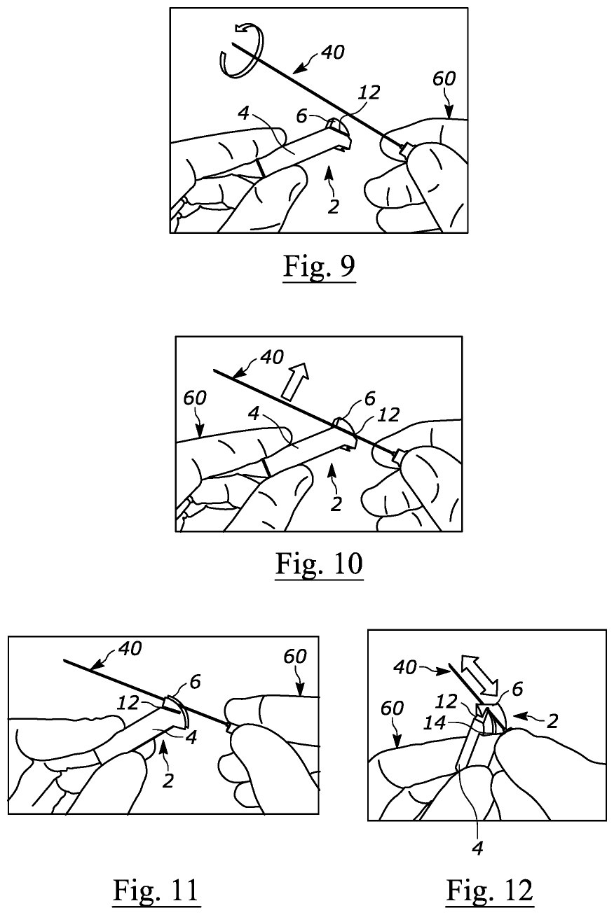

Methods of using the device 2 to bend a medical or surgical instrument (e.g., the needle 40) will now be described with reference to FIGS. 9-16. Any one or combination of the method steps described below may be performed in a point-of-care setting including, for example, an operating room, a hospital room, a doctor's office, a field hospital, and the like, or any other point-of-use setting. Furthermore, the order or sequence of any method steps described below may be varied or re-sequenced in any manner according to alternative embodiments. Moreover, any one or combination of the method steps described below may be omitted in alternative embodiments.

FIGS. 9-14 illustrate a method of using the device 2 to bend the needle 40 according to one embodiment of the present disclosure. As an initial step (not shown in FIGS. 9-14), the device 2 may be cleaned so that it has an sterile or aseptic condition. This step may be omitted in embodiments where the device 2 is stored inside of sterile or aseptic packaging, in which case, the user may remove the device 2 from the packaging and begin to use the device 2 without cleaning it. Alternatively, the user may clean the device 2 after removing it from the sterile or aseptic packaging prior to use.

As shown in FIG. 9, prior to or after inserting the needle 40 into the bending die 6 and prior to or simultaneous with bending the needle 40, a user 60 with his or her hand may rotate the needle 40 about the longitudinal axis A of the needle 40 to position the bevel 46 in a predetermined bevel position. As an example, the predetermined bevel position may correspond to a desired rotational position of the bevel 46 with respect to the to-be-formed bend. As a more specific example, the predetermined bevel position may correspond to the bevel 46 facing in a generally downwards direction (or any other direction) and end of the needle 40 not including the bevel 46 being bent is a generally downwards direction.

Next, if it has not already been done, the user 60 may laterally insert the needle 40 into the bending die 6, as shown in FIG. 10. As an example, laterally inserting the needle 40 into the bending die 6 may involve moving (e.g., linearly moving) the needle 40 in a direction that is perpendicular, non-parallel to, or otherwise transverse to the longitudinal axis A of the needle 40 toward the bending die 6 and into the first recess 12 of the bending die 6. The tip of the needle 40 including the bevel 46 may remain outside of the bending die 6 during this step, as well as throughout a remainder of the bending process. Accordingly, the tip of the needle 40 may be protected from inadvertent contact with the bending die 6 and/or may remain visible to the user 60 throughout the bending process so that the user 60 is able to, for example, maintain the bevel 46 in the predetermined bevel position.

After laterally inserting the needle 40 into the first recess 12, the user 60 may further move the needle 40 laterally so that the needle 40 moves through the first recess 12 into the second recess 14, as shown in FIGS. 11 and 12. When this movement is complete, the needle 40 may extend through the opening 26 in the fourth wall 24 of the bending die 6 such that, for example, the end of the needle 40 including the bevel 46 is positioned outside of the bending die 6. After or simultaneous with moving the needle 40 into the second recess 14, the user 60 may translate the needle 40 with respect to the bending die 6 to position the needle 40 in a first predetermined bending position with respect to the bending die 6. As an example, the user 60 may linearly move the needle 40 in an axial direction along the longitudinal axis A of the needle 40 in order to position the needle 40 in the first predetermined bending position with respect to the bending die. The first bending position may result in the bending die 6 forming a bend in the needle 40 at a desired position along the longitudinal axis A of the needle 40.

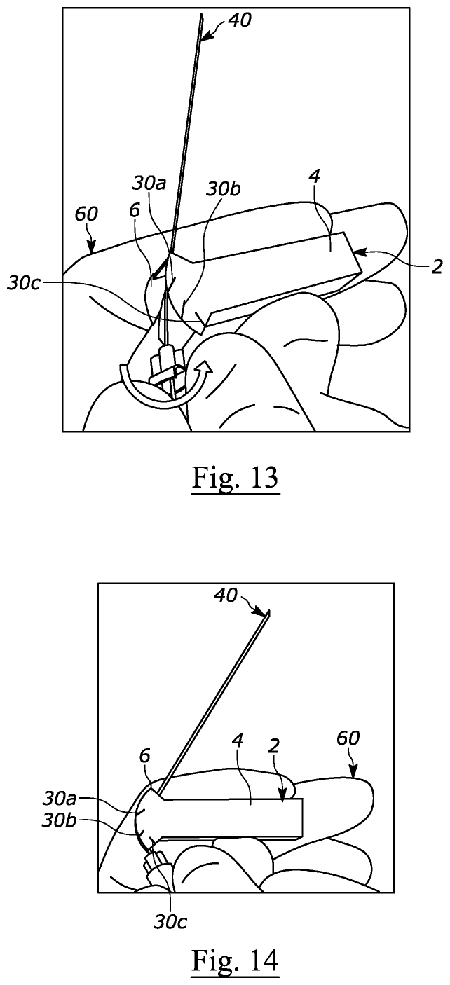

Next, the user 60 may rotate the bending die 6 with respect to the needle 40 to press the needle 40 against the bending surface 16 to form a bend in the needle 40, as shown FIGS. 13 and 14. This step may involve rotating the bending die 6 (e.g., using the handle 4) with one hand while using the other hand to hold an end of the needle 40 stationary, or rotating an end of the needle 40 in one hand while using the other hand to hold the bending die 6 stationary, or rotating an end of the needle 40 in one rotational direction with one hand while using the other hand to rotate the bending die 6 (e.g., using the handle 4) in the opposite rotational direction. The needle 40 may conform to the shape of the bending surface 16 as a result of this relative movement. Furthermore, as a result of this relative movement, the needle 40 may be pressed against the abutment surface 24b of the fourth wall 24 of the bending die 6. The abutment surface 24b may keep the needle 40 pressed against the bending surface 16 by preventing the tip end of the needle 40 from moving away from the bending die 6. Thus, the abutment surface 24b may alleviate the user 60 from having the hold the tip end of the needle 40 in his or her hand, thereby reducing the possibility of the user 60 accidentally sticking himself/herself with the sharpened tip of the needle 40. Furthermore, in bending the needle 40, the user 60 may use one or more of the markings 30a-30c as a visual reference to shape the bend with a desired curvature and/or angle.

After a first bend is formed in the needle 40, the user 60 may form additional bend(s) in the needle 40 without removing the needle 40 from the bending die 6. For example, after creating the first bend, the user 60 may translate the needle 40 with respect to the bending die 6 to position the needle 40 in a second predetermined bending position with respect to the bending die 6. Subsequently, the user 60 may rotate the bending die 6 with respect to the needle 40 to create a second bend in the needle 40 at a second desired position along the longitudinal axis A of the needle 40.

Once the desired bend(s) have been created in the needle 40, the needle 40 may be removed from the bending die 6 via the first recess 12 and/or via the opening 26. Thereafter, the bending die 6 may be reused to bend one or more additional needles and/or other medical or surgical instrument(s). In some embodiments, the bending die 6 may be reused to bend one or more additional needles with identical or substantially similar bend(s) as the needle 40.

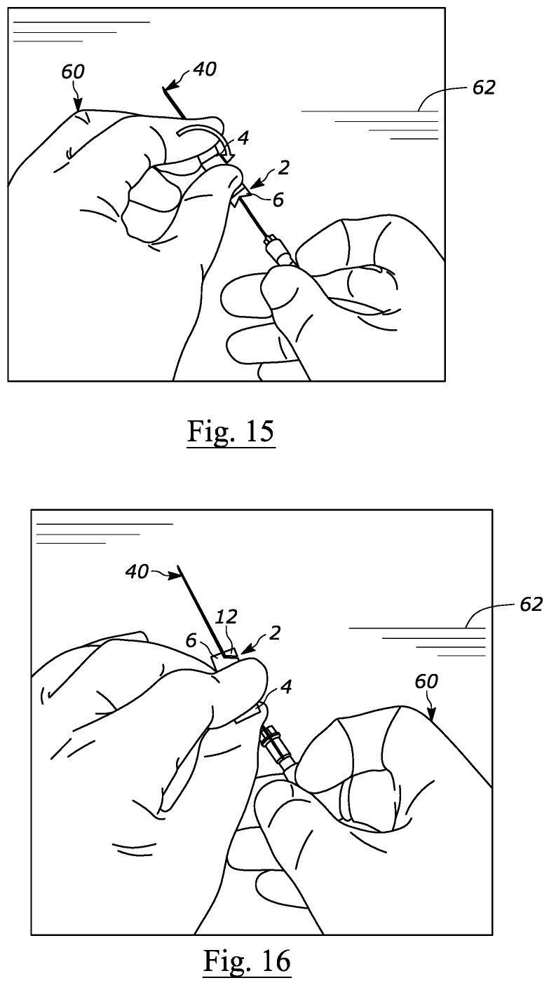

Referring now to FIGS. 15 and 16, an alternative version of the bending step illustrated in FIGS. 13 and 14 will be described. In FIGS. 15 and 16, an external object 62 is used to assist in bending the needle 40. The external object 62 may be any object including a generally planar surface including, for example, a surgical tray, a surgical table, a tabletop, a countertop, and the like. Prior to or simultaneous with bending the needle 40, the user 60 may position the bending die 6 such that one or more of the rolling surfaces 20a, 22a, and 23a are in contact with the external object 62 and such that the needle 40 (or at least the end of the needle 40 including the tip) rests (e.g., lies flat) against the external object 62. Subsequently, with a portion of the needle 40 positioned in the second recess 14, the user 60 may roll the rolling surfaces 20a, 22a, and/or 23a against the external object 62. As an example, the bending die 6 may be rotated in a direction such that the fourth wall 24 moves away from the external object 62 and such that the rolling surfaces 20a, 22a, and/or 23a roll against the external object 62. As a result of this motion, the abutment surface 24b of the fourth wall 24 may pull a portion of the needle 40 away from the external object 62, thereby causing the portion of the needle 40 in the second recess 14 to be pushed against the bending surface 16. This, in turn, may cause the portion of the needle 40 in the second recess 14 to conform to the shape of the bending surface 16, thereby forming the bend in the needle 40. During the rolling process, the user 60 may grip the end of the needle 40 that does not include the tip, but this is not required. Use of the external object 62 to form one or more bends in the needle 40 may have advantageous effects in terms of stability and/or mechanical leverage in certain situations.

From the foregoing, it can be seen that the present disclosure advantageously provides devices and methods facilitating the customization of a medical or surgical instrument by allowing one to create one or more desired bends in the medical or surgical instrument. The presently disclosed devices and methods can form the bends precisely, in a reproducible manner, and without compromising the structural integrity or cleanliness of the medical or surgical instrument. Additional benefits and advantages will be apparent to one of ordinary skill in the art having reviewed the present disclosure.

### EXEMPLARY EMBODIMENTS

Exemplary embodiments provided in accordance with the presently disclosed subject matter include, but are not limited to, the claims and the following embodiments:

Embodiment 1: A device for bending a medical or surgical instrument, the device comprising: a handle; and a bending die comprising: a first end coupled to the handle, a second end, and a first recess formed at least partially in the second end, the first recess being configured to permit at least a portion of the medical or surgical instrument to be inserted laterally into the bending die.

Embodiment 2: The device of Embodiment 1, further comprising a second recess formed at least partially in the second end of the bending die, wherein the first recess cooperates with the second recess such that the medical or surgical instrument can be inserted into the second recess by way of the first recess.

Embodiment 3: The device of any one of Embodiments 1 or 2, the second end of the bending die comprising a bending surface disposed at least partially at a bottom of the second recess, wherein the bending surface is configured to impart at least one bend to the medical or surgical instrument during use of the device.

Embodiment 4: The device of Embodiment 3, wherein at least a portion of the bending surface is convex.

Embodiment 5: The device of any one of Embodiments 3 or 4, wherein the second end of the bending die comprises a rolling surface configured to roll against an external object to assist in causing the medical or surgical instrument to conform to a shape of the bending surface during use of the device.

Embodiment 6: The device of Embodiment 5, wherein at least a portion of the rolling surface is convex.

Embodiment 7: The device of any one of Embodiments 5 or 6, wherein at least a portion of the rolling surface has a curvature corresponding to a curvature of at least a portion of the bending surface.

Embodiment 8: The device of any one of Embodiments 2 to 7, wherein the first recess intersects with the second recess at an angle that is less than or equal to 90 degrees.

Embodiment 9: The device of any one of Embodiments 2 to 8, wherein the bending die comprises a first wall, a second wall, a third wall, and a fourth wall extending between the first and second walls.

Embodiment 10: The device of Embodiment 9, wherein at least a portion of the first recess is disposed between the first wall and the third wall, and at least a portion of the second recess is disposed between the second wall and the third wall.

Embodiment 11: The device of any one of Embodiments 9 or 10, further comprising an opening formed at least partially in the fourth wall and communicating with the first recess and/or the second recess.

Embodiment 12: The device of any one of Embodiments 1 to 11, wherein the handle and the bending die are integrally formed to define a single unitary structure.

Embodiment 13: The device of any one of Embodiments 1 to 12, wherein the first recess is configured to permit the medical or surgical instrument to be inserted into the bending die in a direction transverse to a longitudinal axis of the medical or surgical instrument.

Embodiment 14: The device of any one of Embodiments 1 to 13, wherein the bending die comprises a plurality of markings for assisting a user in bending the medical or surgical instrument.

Embodiment 15: The device of any one of Embodiments 1 to 14, wherein the bending die is provided in a sterile or aseptic condition.

Embodiment 16: The device of any one of Embodiments 1 to 15, wherein the handle is configured to held in at least one hand of a user.

Embodiment 17: The device of any one of Embodiments 1 to 16, wherein the medical or surgical instrument comprises a hypodermic needle.

Embodiment 18: A method of bending a medical or surgical instrument, the method comprising: providing a device having a handle and a bending die; laterally inserting the medical or surgical instrument into the bending die; and rotating the bending die with respect to the medical or surgical instrument to press the medical or surgical instrument against the bending die to form a first bend in the medical or surgical instrument.

Embodiment 19: The method of Embodiment 18, wherein rotating the bending die with respect to the medical or surgical instrument to press the medical or surgical instrument against the bending die to form the first bend in the medical or surgical instrument comprises rolling the bending die against an external object.

Embodiment 20: The method of any one of Embodiments 18 or 19, further comprising, after or simultaneous with laterally inserting the medical or surgical instrument into the bending die and prior to or simultaneous with forming the first bend, translating the medical or surgical instrument with respect to the bending die to position the medical or surgical instrument in a first predetermined bending position with respect to the bending die.

Embodiment 21: The method of Embodiment 20, further comprising, after forming the first bend in the medical or surgical instrument: (a) translating the medical or surgical instrument with respect to the bending die to position the medical or surgical instrument in a second predetermined bending position with respect to the bending die; and (b) subsequent to or simultaneous with (a), further rotating the bending die with respect to medical or surgical instrument to press the medical or surgical instrument against the bending die to form a second bend in the medical or surgical instrument.

Embodiment 22: The method of Embodiment 21, wherein at least a portion of the medical or surgical instrument remains disposed within the bending die during an entire time between forming the first bend and forming the second bend.

Embodiment 23: The method of any one of Embodiments 18 to 22, wherein laterally inserting the medical or surgical instrument into the bending die comprises translating the medical or surgical instrument in a direction transverse to a longitudinal axis of the medical or surgical instrument.

Embodiment 24: The method of any one of Embodiments 18 to 23, wherein the medical or surgical instrument comprises a hypodermic needle.

Embodiment 25: The method of Embodiment 24, further comprising, prior to or simultaneous with forming the first bend, rotating the hypodermic needle about a longitudinal axis of the hypodermic needle to position a bevel of the hypodermic needle in a predetermined bevel position with respect to the to-be-formed first bend.

Embodiment 26: The method of any one of Embodiments 18 to 25, wherein a user operates the handle to manually rotate the bending die with respect to the medical or surgical instrument.

Embodiment 27: The method of any one of Embodiments 18 to 26, further comprising using the medical or surgical instrument in a medical or surgical procedure performed on a patient substantially immediately after bending the medical or surgical instrument.

Embodiment 28: The method of any one of Embodiments 18 to 27, wherein the device is the device according to any one of Embodiments 1 to 17.

While the present disclosure has been described in connection with various embodiments, it will be understood that the present disclosure is capable of further modifications. The present disclosure is intended to cover any variations, uses, or adaptations of the disclosed subject matter following, in general, the principles of the present disclosure, and including such departures from the present disclosure as, within the known and customary practice within the art to which the present disclosure pertains.

1. A device for bending a medical or surgical instrument, the device comprising:
a handle; and
a bending die comprising:
a first end coupled to the handle,
a second end, and
a first recess formed at least partially in the second end, the first recess being configured to permit at least a portion of the medical or surgical instrument to be inserted laterally into the bending die.

2. The device of claim 1, further comprising a second recess formed at least partially in the second end of the bending die, wherein the first recess cooperates with the second recess such that the medical or surgical instrument can be inserted into the second recess by way of the first recess.

3. The device of claim 1, the second end of the bending die comprising a bending surface disposed at least partially at a bottom of the second recess, wherein the bending surface is configured to impart at least one bend to the medical or surgical instrument during use of the device.

4. The device of claim 3, wherein at least a portion of the bending surface is convex.

5. The device of claim 3, wherein the second end of the bending die comprises a rolling surface configured to roll against an external object to assist in causing the medical or surgical instrument to conform to a shape of the bending surface during use of the device.

6. The device of claim 5, wherein at least a portion of the rolling surface is convex.

7. The device of claim 5, wherein at least a portion of the rolling surface has a curvature corresponding to a curvature of at least a portion of the bending surface.

8. The device of claim 2, wherein the first recess intersects with the second recess at an angle that is less than or equal to 90 degrees.

9. The device of claim 2, wherein the bending die comprises a first wall, a second wall, a third wall, and a fourth wall extending between the first and second walls, and wherein at least a portion of the first recess is disposed between the first wall and the third wall, and at least a portion of the second recess is disposed between the second wall and the third wall.

10. (canceled)

11. (canceled)

12. The device of claim 1, wherein the handle and the bending die are integrally formed to define a single unitary structure.

13. The device of claim 1, wherein the first recess is configured to permit the medical or surgical instrument to be inserted into the bending die in a direction transverse to a longitudinal axis of the medical or surgical instrument.

14. The device of claim 1, wherein:
the bending die comprises a Plurality of markings for assisting a user in bending the medical or surgical instrument;
the bending die is provided in a sterile or aseptic condition;
the handle is configured to held in at least one hand of a user; and/or
the medical or surgical instrument comprises a hypodermic needle.

15. (canceled)

16. (canceled)

17. (canceled)

18. A method of bending a medical or surgical instrument, the method comprising:
providing a device having a handle and a bending die;
laterally inserting the medical or surgical instrument into the bending die; and
rotating the bending die with respect to the medical or surgical instrument to press the medical or surgical instrument against the bending die to form a first bend in the medical or surgical instrument.

19. The method of claim 18, wherein rotating the bending die with respect to the medical or surgical instrument to press the medical or surgical instrument against the bending die to form the first bend in the medical or surgical instrument comprises rolling the bending die against an external object.

20. The method of claim 18, further comprising, after or simultaneous with laterally inserting the medical or surgical instrument into the bending die and prior to or simultaneous with forming the first bend, translating the medical or surgical instrument with respect to the bending die to position the medical or surgical instrument in a first predetermined bending position with respect to the bending die.

21. The method of claim 20, further comprising, after forming the first bend in the medical or surgical instrument:
(a) translating the medical or surgical instrument with respect to the bending die to position the medical or surgical instrument in a second predetermined bending position with respect to the bending die; and
(b) subsequent to or simultaneous with (a), further rotating the bending die with respect to medical or surgical instrument to press the medical or surgical instrument against the bending die to form a second bend in the medical or surgical instrument.

22. (canceled)

23. The method of claim 18, wherein laterally inserting the medical or surgical instrument into the bending die comprises translating the medical or surgical instrument in a direction transverse to a longitudinal axis of the medical or surgical instrument.

24. The method of claim 18, wherein the medical or surgical instrument comprises a hypodermic needle.

25. The method of claim 24, further comprising, prior to or simultaneous with forming the first bend, rotating the hypodermic needle about a longitudinal axis of the hypodermic needle to position a bevel of the hypodermic needle in a predetermined bevel position with respect to the to-be-formed first bend.

26. The method of claim 18, wherein a user operates the handle to manually rotate the bending die with respect to the medical or surgical instrument, and wherein the method further comprises using the medical or surgical instrument in a medical or surgical procedure performed on a Patient substantially immediately after bending the medical or surgical instrument.

27. (canceled)

28. (canceled)
